(19) United States
(12) Patent Application Publication
(10) Pub. No.: US 2026/0243167 A1
(43) Pub. Date: Aug. 20, 2026

(54) SAMPLING DEVICE AND ONLINE SURVEYING AND MAPPING SYSTEM

(71) Applicants: CHINA PETROLEUM & CHEMICAL CORPORATION, Beijing (CN); SINOPEC OILFIELD SERVICE CORPORATION, Beijing (CN); SINOPEC SHENGLI PETROLEUM ENGINEERING CO., LTD, Dongying, Shandong (CN); DRILLING TECHNOLOGY RESEARCH INSTITUTE OF SINOPEC SHENGLI PETROLEUM ENGINEERING CO., LTD, Dongying, Shandong (CN)

(72) Inventors: Yanjun ZHOU, Dongying, Shandong (CN); Zhongshuai CHEN, Dongying, Shandong (CN); Xueliang PEI, Dongying, Shandong (CN); Zhihe LIU, Dongying, Shandong (CN); Kai TAN, Dongying, Shandong (CN); Feng LIU, Dongying, Shandong (CN); Chuanwei ZHAO, Dongying, Shandong (CN); Yanwen WANG, Dongying, Shandong (CN); Guiting WANG, Dongying, Shandong (CN); Haobo LI, Dongying, Shandong (CN)

(21) Appl. No.: 19/143,061

(22) PCT Filed: Dec. 27, 2022
(86) PCT No.: PCT/CN2022/142254

§ 371 Date: Jun. 25, 2025

(51) Int. Cl. E21B 49/10; E21B 47/12; G01N 1/14; G01N 33/28

(52) U.S. Cl. CPC E21B 49/10; E21B 47/12; G01N 1/14; G01N 33/2823

(57) ABSTRACT

A sampling device and an online surveying and mapping system has a ground control unit for transmitting a control command downhole and a downhole unit. The downhole unit includes a housing; a downhole control assembly for receiving the control command from the ground control unit to control sampling and online surveying and mapping operations; a probe assembly configured to draw a sample of a formation; a power system for driving the probe assembly to draw samples; a tank assembly for storing qualified formation samples; a contamination-rate evaluating assembly for determining whether a contamination rate of the sample of the formation is qualified and discharging unqualified sample of the formation while delivering qualified sample of the formation to the tank assembly; and a fluid-parameter measuring assembly for measuring parameters of the sample of the formation in the tank assembly and transmitting measured data to the ground control unit.

### TECHNICAL FIELD

The present invention relates to the field of formation fluid sampling in oil and gas drilling, in particular a sampling device and an online surveying and mapping system.

### TECHNICAL BACKGROUND

It is of great significance to collect representative samples of formation fluid for obtaining geological parameters, such as fluid types and properties, in order to accurately determine the oil and gas content of reservoirs, and to reasonably formulate medium-and long-term development plans for oil and gas fields.

Currently, the acquisition of formation fluid is achieved primarily by lowering a cable sampling tool. The cable sampling tool works by pressing a small padding probe against the wall of a well, adjusting the pressure within the probe sufficiently to break the mudcake sealing, and then pumping the fluid out of the formation into a sealable sampling chamber. After that, the composition of the sample in the sampling chamber is analyzed at the ground, thus determining the content of hydrocarbons.

The technique of cable fluid-sampling has been developed for a long time and is mature now. However, its operation takes up a long time on the drilling rig, and the tool is easy to be stuck due to the mud circulation problems in the sampling procedure. In the meantime, it is also difficult to lower the tool into highly-deviated wells, horizontal wells or extended reach wells. In addition, the cable fluid-sampling is a post-drilling approach, so that the formation has to be opened for a relatively long time, and thus the formation near the well wall is easy to be contaminated by mud. Moreover, the formation fluid has to be removed with formation testing technique for analysis in the laboratory, which often leads to errors, making it difficult to obtain the true component data of the formation fluid, and is costly and time-consuming.

In the conventional cable-based downhole formation fluid sampling devices, formation fluid sample is stored in a sampling cylinder, and a fluid line between the sampling cylinder and the sampling unit is opened or closed via a one-way valve, thus realizing the sampling function. The whole system is regulated by a control unit. However, such cable fluid-sampling tool cannot achieve sampling-while-drilling or online surveying and mapping of fluid performance parameters.

### SUMMARY OF THE INVENTION

Aiming at the above technical problems existing in the prior arts, the present invention proposes a sampling device for sampling-while-drilling of formation fluid. The present invention further proposes an online surveying and mapping system, which can achieve sampling-while-drilling for the formation fluid through a sampling command from the ground, measure a contamination rate of the formation fluid in an online manner, and store qualified formation fluid into a sample tank. At the same time, the system of the present invention can also realize online surveying-and-mapping of the properties of formation fluid and transmit the results to the ground.

The present invention proposes a sampling device, comprising: a housing comprising a body and a sampling drill collar; a sampling assembly arranged in the sampling drill collar, comprising a probe assembly, which includes an external probe support mechanism and an internal probe, wherein the probe support mechanism is provided at a bottom thereof with a push piston, for driving the probe to extend in a radial direction relative the sampling drill collar; and a power assembly and a suction assembly arranged on the body. The power assembly is configured to push the probe outward in the radial direction into a formation via the push piston, and the suction assembly is configured to draw fluid sample from the formation via the probe, and the sampling assembly further comprises a recovery mechanism, for driving the probe to retract after drawing.

According to an improvement of the present invention, a plurality of recovery mechanisms is uniformly arranged around a circumferential direction of the probe. Each of the plurality of recovery mechanisms includes an telescopic rod connected to the probe support mechanism and the probe, a recovery spring being arranged externally around the telescopic rod, wherein an end of the recovery spring is connected to the probe and another end thereof is connected to the probe support mechanism.

According to an improvement of the present invention, the body is provided with a piston chamber, in which a power piston is provided. The piston chamber is filled with hydraulic oil, and has a front end in communication with the push piston, wherein the power piston is moveable in the piston chamber, thereby pumping the hydraulic oil to or away from the push piston.

According to an improvement of the present invention, the power assembly further includes a servo motor connected to the power piston via a screw, wherein the servo motor is configured to drive the screw to rotate, so that the screw, when rotating, drives the power piston to extend or retract in the piston chamber via threads.

According to an improvement of the present invention, the probe is provided therein with a central hole in communication with a flow channel, and the probe is further provided with a filter.

According to an improvement of the present invention, the suction assembly includes a suction pump, which is connected to the central hole of the probe via the flow channel.

According to an improvement of the present invention, the power assembly and the suction assembly are powered by a power supply assembly, which includes a rectifier voltage regulator circuit, a main control circuit, a push drive circuit, a suction drive circuit, and an electromagnetic valve control circuit, wherein the push drive circuit supplies power to the servo motor, and the suction drive circuit supplies power to the suction pump.

According to an improvement of the present invention, an end of the probe assembly is provided with a differential pressure sensor connected to the push drive circuit.

According to a further aspect of the present invention, an online surveying-and-mapping system is proposed, comprising: a ground control unit for transmitting a control command from ground to downhole; and a downhole unit. The downhole unit comprises: a sampling device as mentioned above; a downhole control assembly, for receiving the control command from the ground control unit to control sampling and online surveying and mapping operations; a tank assembly, for storing qualified formation samples; a contamination-rate evaluating assembly, for determining whether a contamination rate of a formation sample is qualified, and discharging unqualified formation sample while delivering qualified formation sample to the tank assembly; and a fluid-parameter measuring assembly, for measuring parameters of the formation sample in the tank assembly and transmitting measured data to the ground control unit.

According to an improvement of the present invention, the body is provided at an upper end thereof with an instrument compartment case and an upper case, for installing the downhole control assembly, and at a lower end thereof with the sampling drill collar, wherein the sampling drill collar is provided at a lower end thereof with a sample-measuring drill collar, for installing the fluid-parameter measuring assembly.

According to an improvement of the present invention, the sampling drill collar is provided with a spiral wing having an outer diameter slightly smaller than wellbore diameter, wherein the spiral wing is provided with a through-hole slot for installing the probe assembly.

According to an improvement of the present invention, the suction assembly and the contamination-rate evaluating assembly are formed into one piece and connected to the suction pump via a suction line. The suction pump is configured to provide a negative pressure to the probe via the flow channel for drawing the formation fluid, which flows to the contamination-rate evaluating assembly through the flow channel and the suction line.

According to an improvement of the present invention, a sidewall of the body is further provided with a sidewall hole communicating the contamination-rate evaluating assembly with an external annulus, wherein the suction pump pumps the formation sample drawn by the probe assembly to the contamination-rate evaluating assembly, which discharges the unqualified formation sample through the sidewall hole and delivers qualified formation sample to the tank assembly.

According to an improvement of the present invention, the contamination-rate evaluating assembly is connected to the tank assembly via the suction line, the suction pump, the flow channel, a first pipeline and a second pipeline, and an electromagnetic valve or a one-way valve is arranged in each of the flow channel, the first pipeline and the second pipeline.

According to an improvement of the present invention, the tank assembly and the fluid-parameter measuring assembly are located inside the sample-measuring drill collar, and the fluid-parameter measuring assembly is configured to measure parameters of the formation sample and upload the parameters to the ground control unit.

According to an improvement of the present invention, the fluid-parameter measuring assembly is connected to an adapter connected to a switch valve via the suction line.

The present invention can achieve the following advantages over the prior arts.

The sampling device of the present invention is extendable and retractable, so that the probe can extend to a suitable position in all downhole circumstances, and thus abut against the formation to draw the formation sample. The sampling assembly is arranged on a sampling drill collar, while the power assembly and the drawing assembly are both arranged on the housing body. The powering piston is arranged axially and has a relatively long travel distance. The extension and retraction of the probe can be precisely controlled by the servo motor.

The online surveying and mapping system of the present invention can achieve sampling-while-drilling for the formation fluid through a sampling command from the ground, measure the contamination rate of the formation fluid in an online manner, and store the qualified formation fluid into the sample tank. At the same time, the system of the present invention can also realize online surveying and mapping of the properties of formation fluid and transmit the results to the ground.

With the sampling device and the online surveying and mapping system of the present invention, sampling-while-drilling for formation fluid can be achieved, and critical properties of the formation fluid can be measured in an online manner. Compared with the cable-based sampling tool, it is easier to obtain original formation fluid according to the present invention, because in the present invention the formation is opened for a relatively short time during sampling, and the near-wall region of the wellbore is less contaminated by the mud intrusion filtrate. In addition, in environments where conventional cable sampling is restricted, such as in highly-deviated wells, horizontal wells, extended reach wells or the like, the present invention can be applied to realize sampling of the formation fluid.

### BRIEF DESCRIPTION OF THE DRAWINGS

Preferred embodiments of the present invention will be described in detail below in conjunction with the accompanying drawings. In the drawings:

FIG. 1 schematically shows the principle of a sampling device according to one embodiment of the present invention;

FIG. 2 schematically shows a structure of the sampling device according to one embodiment of the present invention, showing a structure of the probe assembly;

FIG. 3 schematically shows a structure of an online surveying and mapping system according to one embodiment of the present invention;

FIG. 4 schematically shows a structure of a downhole unit according to one embodiment of the present invention in a sectional view;

FIG. 5 schematically shows a structure of an external housing according to one embodiment of the present invention;

FIG. 6 schematically shows a structure of a power assembly according to one embodiment of the present invention;

FIG. 7 schematically shows a structure of a body according to one embodiment of the present invention; and

FIG. 8 is a sectional view of FIG. 7 along line A-A.

In the accompanying drawings, the same member is indicated by the same reference sign. The accompanying drawings are not necessarily drawn to actual scale.

### List of Reference Signs

1 ground control unit; 2 housing; 3 power supply assembly; 4 sampling assembly; 5 power system; 6 contamination-rate evaluating assembly; 7 tank assembly; 8 fluid-parameter measuring assembly; 9 downhole control assembly; 10 formation; 11 ground pulse transmitter; 12 mud pit; 13 mud pump; 21 body; 22 instrument compartment case; 23 upper case; 24 sampling drill collar; 25 sample-measuring drill collar; 26 dual male connector; 211 first cover; 212 first open slot; 213 second open slot; 251 spiral wing; 252 through-hole slot; 31 slip ring; 32 O-ring; 33 push drive circuit; 34 suction drive circuit; 331 push drive circuit cable; 332 push drive circuit connector; 333 first carbide impact-resistant ring; 334 push drive circuit connector line; 335 multi-core connector; 341 instrument compartment conduit; 342 suction drive circuit connector; 343 suction drive circuit cable; 344 second carbide impact-resistant ring; 345 suction drive circuit connector line; 41 probe assembly; 42 filter; 43 push piston; 44 recovery mechanism; 45 differential pressure sensor; 47 hydraulic orifice; 51 power assembly; 52 suction assembly; 511 servo motor; 512 coupling; 513 reducer; 514 bearing pack; 515 screw; 516 power piston; 517 piston chamber; 518 first pressure sensor; 519 liquid channel; 521 suction pump; 522 suction line; 523 sidewall hole; 524 switching valve; 525 flow channel; 526 second pressure sensor; 527 upper line; 71 sample tank; 72 flow pathway; 73 first pipeline; 74 second pipeline; 75 adapter; 76 tank piping; 81 multiport solenoid valve; 82 fluid-parameter measuring assembly pipeline; 110 formation fluid; 111 drilling tool.

### DETAILED DESCRIPTION OF THE INVENTION

In order to enable the technical solutions and advantages of the present invention more clearly understood, exemplary embodiments of the present invention will be described in further detail in conjunction with the accompanying drawings. Obviously, the described embodiments are only a part of embodiments of the present invention, but not all the embodiments. The embodiments and the features in respective embodiments in the present invention may be combined with each other as long as there is no conflict.

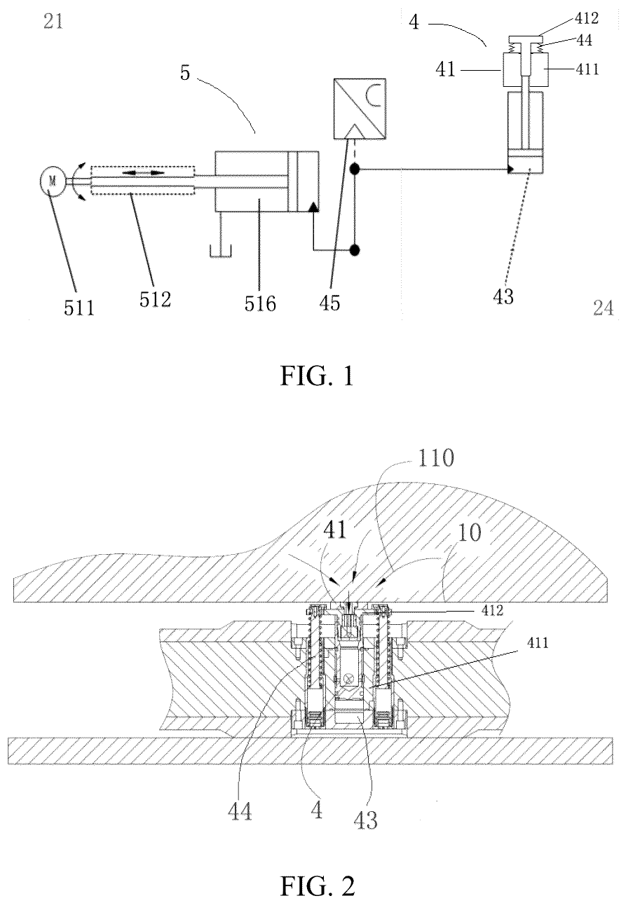

FIG. 1 schematically shows a sampling device according to one embodiment of the present invention. The sampling device comprises a housing 2, which includes a body 21 and a sampling drill collar 24. The sampling drill collar 24 is provided with a sampling assembly 4 comprising a probe assembly 41, as shown in FIG. 2. The probe assembly 41 comprises a probe supporting mechanism 411 fixedly arranged on the sampling drill collar 24, and a probe 412 arranged inside the probe supporting mechanism 411 and configured to be movable therein. The probe supporting mechanism 411 is provided at its bottom with a push piston 43, which can drive the probe to extend forwardly.

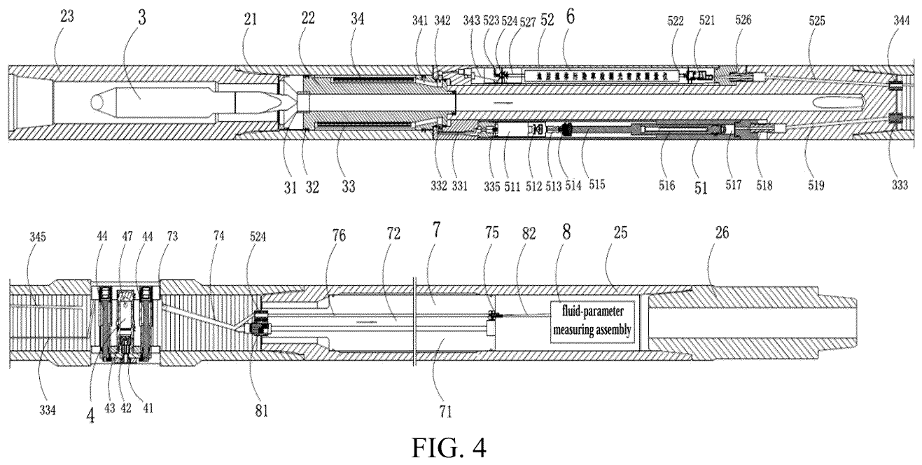

As shown in FIGS. 1 and 4, the body 21 is provided with a power assembly 51 and a suction assembly 52. The push piston 43 is connected to the power assembly 51 through a liquid channel 519. The power assembly 51 can provide power to the push piston 43 to extend and retract. The power assembly 51 pushes the probe 412 to extend outwardly in a radial direction into the formation through the push piston 43, and the suction assembly 52 draws a sample of formation fluid through the probe.

The sampling assembly 4 further comprises a recovery mechanism 44, for driving the probe 412 to retract upon completion of suction.

The probe assembly 41 of the sampling device of the present embodiment is connected to the suction assembly 52. Similar to the power assembly 51, the suction assembly 52 is arranged in an axial direction of the sampling drill collar, and separate from the probe assembly 41.

During operation, a power piston 516 of the power assembly 51 is electrically driven to transmit hydraulic pressure to the push piston 43, which drives the probe to extend outwardly into the formation. A suction pump 512 of the power assembly 51 draws a sample of formation fluid through the probe, and after that, the power piston 516 is retracted so that pressure is released from the push piston 43. In this case, the recovery mechanism 44 drives the probe to retract.

In one embodiment, a plurality of recovery mechanisms 44 is uniformly arranged around an outer edge of the probe 412 for a full circle. In this example, each recovery mechanism 44 comprises a telescopic rod 441 connected to the supporting mechanism 411 and the probe 412 respectively. A recovery spring 442 is arranged outside the telescopic rod. One end of the recovery spring is connected to the probe 412, and the other end thereof is connected to the supporting mechanism. When the push piston 43 pushes the probe to extend, the spring is in an extended state. When pressure is released from the push piston 43, the spring pulls the probe to retract.

After the push piston 43 pushes out the probe and draws a sample from the formation, pressure is released from the push piston 43. Therefore, the probe will retract and restore to its original position under the actions of formation pressure and the recovery mechanism 44. The recovery mechanism 44 can ensure that the probe retracts smoothly.

In one embodiment, the body 21 is provided with a piston chamber 517, in which the power piston 516 is arranged. The piston chamber 517 is filled with hydraulic oil, and is connected to the push piston 43 at its front end. The power piston 516 can move in the piston chamber 517, so as to pump the hydraulic oil to or away from the push piston 43.

In this embodiment, the power piston 516 is arranged at a position different from the push piston 43. The power piston 516 is arranged along the axial direction of the sampling drill collar, so that it has a large range of movement and can provide a large hydraulic pressure to the push piston 43. The power piston 516 is electrically driven to move in the axial direction, thereby pushing the hydraulic oil in the piston chamber to a region under the push piston, which will move forward under the action of the hydraulic pressure, and thus drive the probe to extend forward. The power assembly 51 is arranged along the axial direction of the body and can have a large length, which, on the one hand, can pump more hydraulic fluid into the probe assembly 41, and on the other hand, can also accurately control the extended or retracted length of the probe 412 based on the travel distance of the power piston 516.

In a preferred embodiment, the power assembly 51 further comprises a servo motor 511, which is connected to the power piston 516 via a screw 515. The servo motor 511 can drive the screw 515 to rotate, wherein the screw 515 drives the power piston 516 to extend or retract within the piston chamber 517 through threads when the screw 515 rotates, so as to pump the hydraulic oil within the piston chamber 517 to or away from the push piston 43.

Preferably, the servo motor 511 is indirectly connected to the screw 515 through a connecting shaft, a reducer 513, and a bearing pack 514, and can drive the screw 515 to rotate clockwise or counterclockwise for driving the power piston 516 to extend or retract. In this manner, hydraulic oil inside the piston chamber 517 of the power piston 516 will be delivered to the bottom of the suction probe or drawn away therefrom, thus driving the suction probe to extend or retract, so that the probe can be controlled to be in contact with or separate from the formation 10.

In one embodiment, the probe is provided with a center hole formed therein, which is connected to a flow channel 525. The probe is further provided with a filter 42, for filtering impurities or large particles in the sample from the formation.

Preferably, the suction assembly 52 comprises a suction pump 512 connected to the probe through the flow channel 525.

In one embodiment, the power assembly 51 and the suction assembly 52 are supplied with power through a power supply assembly 3, which comprises a rectifier regulator circuit, a master control circuit, a push drive circuit 33, a suction drive circuit 34, and a solenoid control circuit, wherein the push drive circuit 33 supplies power to the servo motor 511 and the suction drive circuit 34 supplies power to the suction pump 512.

The probe assembly 41 is provided at an end thereof with a differential pressure sensor 45 connected to the push drive circuit 33. Specifically, the differential pressure sensor 45 is arranged on a line connecting the probe assembly 41 to the suction assembly. Thus it can determine whether the probe of the probe assembly 41 is desirably extended through detecting the differential pressure. The push drive circuit 33 is connected through a push drive circuit connector 332 to a push drive circuit cable 331, which is connected to the servo motor 511 through a multi-core connector 335.

The power piston 516 extends or retracts to drive the hydraulic oil in the piston chamber 517 to or away from the push piston 43, thus controlling the extension or retraction of the push piston 43. In this manner, the extension and retraction of the probe assembly can be controlled. The differential pressure sensor 45 can accurately capture the output pressure. In this procedure, a downhole control assembly 9 is provided to record the number of revolutions of the motor through feedback from an encoder, calculate the travel distance of the piston and the final extension length of the probe. On this basis, it can determine whether the probe assembly is desirably extended in combination with the output pressure of the system.

In accordance with another aspect of the present invention, an online surveying and mapping system is further proposed.

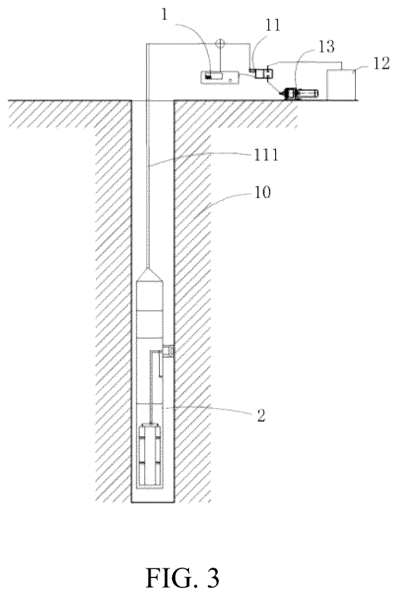

FIG. 3 schematically shows an online surveying and mapping system according to an embodiment of the present invention, comprising a ground control unit 1 and a downhole unit. The ground control unit 1 is arranged on the ground to control operations of the downhole unit through transmitting control commands from the ground to downhole.

The downhole unit is lowered into the well along with a drilling tool 111 for downhole sampling and online surveying and mapping. As shown in FIGS. 3 and 4, the downhole unit comprises a housing 2, which is attached to the drilling tool 111 for protecting other components. The housing 2 is provided therein with: a downhole control assembly 9, for receiving control commands from the ground control unit 1 to control sampling and online surveying and mapping; a sampling assembly 4, configured to draw a sample of the formation 10; a power portion 5, for providing power for extension or retraction of the sampling assembly 4 to draw the sample; a tank assembly 7, for storing qualified samples of the formation 10; a contamination-rate evaluating assembly 6, for determining whether the contamination rate of the samples of the formation 10 is qualified or not, and discharging unqualified samples of the formation 10 while delivering qualified samples of the formation 10 into the tank assembly 7; and a fluid-parameter measuring assembly 8, for measuring parameters of samples of the formation 10 in the tank assembly 7 and transmitting measured data to the ground control unit 1.

When the online surveying and mapping system according to this embodiment is used, the downhole unit is lowered into the well along with the drilling tool 111. An operator sends a control command to the downhole unit through the ground control unit 1, so that the downhole control assembly 9 controls the power portion 5 to provide power to the probe assembly 4, which can extend to contact the formation 10 for drawing a sample of the formation 10. The sample of the formation 10 obtained is detected by the contamination-rate evaluating assembly 6. If the sample is qualified, it is delivered to the tank assembly 7, and the fluid-parameter measuring assembly performs online surveying and mapping on sample of the formation 10; if not, it is discharged into the annulus.

Through sending sampling commands from the ground by the system of this embodiment, sampling-while-drilling of the formation fluid can be realized, and online measurement on the contamination rate of the formation fluid 110 can be conducted, wherein qualified formation fluid 110 is stored in the sample tank 7. At the same time, the system of the present invention also realizes online surveying-and-mapping of properties of the formation fluid and transmission of the results to the ground.

With the system of the present embodiment, sampling-while-drilling for formation fluid can be achieved, and critical properties of the formation fluid can be measured in an online manner. Compared with the cable-based sampling tool, it is easier to obtain original formation fluid according to the present invention, because in the present invention the formation is opened for a relatively short time during sampling, and the near-wall region of the wellbore is less contaminated by the mud intrusion filtrate. In addition, in environments where conventional cable sampling is restricted, such as in highly-deviated wells, horizontal wells, extended reach wells or the like, the system of the present invention can be applied to realize sampling of the formation fluid.

The sampling-while-drilling technique proposed by the present embodiment can rapidly collect formation fluid 110 with low or no contamination when the formation is just opened, which takes shorter operation time while obtaining more reliable data on the formation compared with the cable-based fluid sampling.

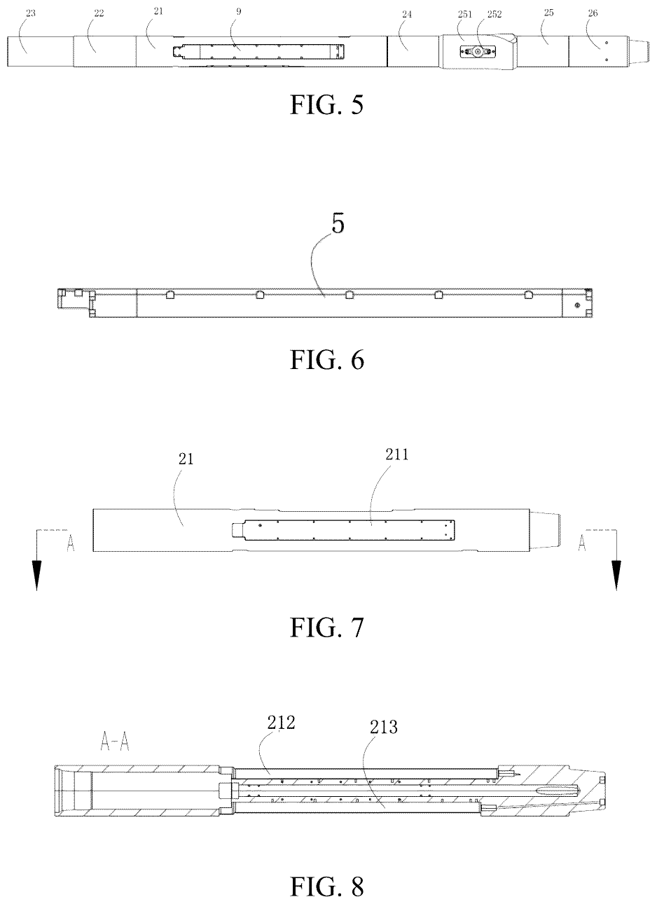

In one embodiment, as shown in FIG. 5, the housing 2 comprises a body 21 for installation of the power portion 5. The body 21 is provided with an instrument compartment case 22 and an upper case 23 at an upper end thereof, for installation of the downhole control assembly 9 and the power portion 5, respectively. The sampling drill collar 24 is provided at a lower end of the body 21, for installation of the probe assembly 4. At the lower end of the sample collar 24 there is a sample-measuring drill collar 25, for installation of the fluid parameter-measuring assembly 8. A double-male connector 26 is arranged at a bottom end of the sample-measuring drill collar 25. The upper case 23, the instrument compartment case 22, the body 21, the sample drill collar 24, the sample-measuring drill collar 25 and the double-male connector 26 are connected with each other via threads.

The housing 2 can be divided into multiple sections, which are connected to each other via threads. In this manner, a firm connection with satisfactory sealing effect can be achieved, and in the meantime, such structure facilitates installation and disassembly of components within respective sections. When assembling various components, they can be installed on corresponding sections of the housing 2. For example, the downhole control assembly 9 and the power portion 5 are installed on the instrument compartment case 22 and the upper case 23 respectively, the probe assembly 4 is installed on the sampling drill collar, and the tank assembly 7 and the fluid-parameter measuring assembly 8 are installed in the sample-measuring drill collar 25. Then, the upper case 23, the instrument compartment case 22, the body 21, the sampling drill collar 24, the sample-measuring drill collar 25, and the double male connector 26 are threaded together to complete the assembly.

In one embodiment, as shown in FIG. 4, the power supply assembly 3, which may be a mud generator or a high capacity battery pack, is arranged in the upper case 23 or the instrument compartment case 22. The power supply assembly 3 is connected via wires to other units requiring power supply. In this embodiment, the power supply assembly 3 includes the rectifier regulator circuit, the main control circuit, the push drive circuit 33, the suction drive circuit 34, and the solenoid valve control circuit. The push drive circuit 33 is configured to power and control the power assembly 51, and the suction drive circuit 34 is configured to power and control the suction assembly 52 and the contamination-rate evaluating assembly 6. The solenoid valve control circuitry is configured to power and control the tank assembly 7 and the fluid-parameter measuring assembly 8.

An instrument compartment carrier is arranged in and secured to the instrument compartment case 22, and is sealed by an O-ring 32. An upper portion of the instrument compartment carrier is connected to the power supply assembly 3 through a slip ring 31, which ensures electrical connection during rotation or sliding. The power supply assembly 3 can ensure the supply of electrical energy to various components of the downhole unit, thus guaranteeing the sampling and online surveying and mapping operations to be performed smoothly.

In one embodiment, as shown in FIG. 6, the body 21 is provided with a first open slot 212 and a second open slot 213 for receiving the power assembly 51 and the suction assembly 52 of the power portion 5, respectively, and is sealed by a first cover 211 (shown in FIG. 7) and a second cover (arranged opposite to the first cover 211 but not shown in FIG. 7). The power assembly 51 is totally placed in the first open slot 212 of the body 21, and pressed therein by the first cover 211 via threads. The power assembly 51 provides power to the probe assembly 4 so that it can extend or retract to be in contact with the formation 10, and the suction assembly 52 provides power to the probe assembly 4 so that it can draw the sample of the formation 10.

When drawing a sample, the probe assembly 4 should reach into the formation 10 and then draw formation fluid 110 from the formation 10 as the sample. During this procedure, the power assembly provides power for the probe assembly 4 to extend or retract, while the suction assembly 52 provides power for the probe assembly 4 to draw the sample.

Preferably, the power assembly 51 in the power portion 5 provides power for the probe assembly 4 to extend or retract through hydraulic pressure, so that the probe assembly 4 can extend into the formation 10, and then the suction assembly 52 controls the probe assembly 4 to draw the sample of the formation 10, thus completing the sample collection. After that, the power assembly 51 controls the probe assembly 4 to retract.

In one embodiment, as shown in FIG. 5, the sampling drill collar 24 is provided with a spiral wing 251 having an outer diameter slightly smaller than the diameter of the wellbore. The spiral wing 251 is provided with a through-hole slot 252 for mounting the probe assembly 4.

When fluid sampling is performed, the probe assembly 4 is hydraulically driven by the power assembly 51 to push the piston 43 to extend outwardly, with a sealing washer of the probe assembly 41 coming into contact with the inner wall of the formation 10. The other side of the sampling drill collar 24 comes into contact with the inner wall of the formation 10 under the action of the pushing force. After the probe assembly 41 is extended to a preset position, the suction assembly 52 is activated so that the probe of the probe assembly 41 begins to draw the formation fluid 110, thus completing the sampling. After that, the probe assembly 41 is recovered to its initial state under the recovery force provided by the recovery mechanism 44.

The probe assembly 41 is provided at an end thereof with a differential pressure sensor 45 connected to the push drive circuit 33. Specifically, the differential pressure sensor 45 is arranged on a line connecting the probe assembly 41 to the suction assembly. Thus it can determine whether the probe of the probe assembly 41 is desirably extended through detecting the differential pressure. The push drive circuit 33 is connected through a push drive circuit connector 332 to a push drive circuit cable 331, which is connected to the servo motor 511 through a multi-core connector 335.

The power piston 516 extends or retracts to drive the hydraulic oil in the piston chamber 517 to or away from the push piston 43, thus controlling the extension or retraction of the push piston 43. In this manner, the extension and retraction of the probe assembly can be controlled. The differential pressure sensor 45 can accurately capture the output pressure. In this procedure, a downhole control assembly 9 is provided to record the number of revolutions of the motor through feedback from an encoder, calculate the travel distance of the piston and the final extension length of the probe. On this basis, it can determine whether the probe assembly is desirably extended in combination with the output pressure of the system.

In one embodiment, as shown in FIGS. 1 and 6, the power assembly 51 includes the servo motor 511, the coupling 512, the reducer 513, the bearing pack 514, the screw 515, and the power piston 516, which are sequentially connected to each other. The power assembly 51 further comprises the multi-core connector 335, and is connected to the push drive circuit 33 of the power assembly 3 through the push drive cable and the multi-core connector 335, in order to perform the action command from the push drive circuit 33. The power piston 516 provides power to the probe assembly for extension or retraction.

The piston chamber 517 of the power piston 516 is connected to the push piston 43 through the liquid channel 519, in which a first pressure sensor 518 and a first carbide impact-resistant ring 333 are arranged. The first carbide impact-resistant ring 333 is connected to a push drive circuit connector line 334, so that the first pressure sensor 518 is connected to the push drive circuit 33 via the first carbide impact-resistant ring 333 and the push drive circuit connector line 334, for providing feedback concerning the pressure on the power piston 516, based on which the push drive circuit 33 determines the state of the power piston 516.

Preferably, the servo motor 511 is indirectly connected to the screw 515 through a connecting shaft, a reducer 513, and a bearing pack 514, and can drive the screw 515 to rotate clockwise or counterclockwise for driving the power piston 516 to extend or retract. In this manner, hydraulic oil inside the piston chamber 517 of the power piston 516 will be delivered to the bottom of the suction probe or drawn away therefrom, thus driving the suction probe to extend or retract, so that the probe can be controlled to be in contact with or separate from the formation 10.

In one embodiment, as shown in FIG. 4, the suction assembly 52 and the contamination-rate evaluating assembly 6 are formed into one piece to be placed in the second open slot 213 of the body 21 and pressed tightly by the second cover. The combination of the suction assembly 52 and the contamination-rate evaluating assembly 6 is connected through a suction line 522 to a suction pump 521, which is connected to the probe assembly 4 through the flow channel 525, so that the formation fluid 110 can be pumped into the contamination-rate evaluating assembly 6 through the suction action of the suction pump 521.

Preferably, the suction drive circuit 34 is connected to a suction drive circuit connector 342 via an instrument compartment conduit 341, and further to the suction assembly 52 via a suction drive circuit cable 343. The body 21 is further provided with a sidewall hole 523 connecting the contamination-rate evaluating assembly 6 to the external annulus. A switching valve 524 is arranged in the sidewall hole 523 for discharging unqualified samples.

The suction pump 521 draws formation fluid 110 from the formation 10 through the probe assembly 4 via the flow channel 525, and delivers it through the suction line 522 to the contamination-rate evaluating assembly 6. Samples with an unqualified contamination rate are discharged through the sidewall hole 523 into the wellbore annulus after passing through the control valve 524. Samples with qualified contamination rate are discharged through the suction pump 521 into the tank assembly 7 for storage. The suction assembly 52 is equipped with a pressure sensor, for recording the fluid pressure in the suction line 522 at a front end of suction pump 521. The pressure sensor is connected to the suction drive circuit 34 via the suction drive circuit cable 343 and the second carbide impact-resistant ring 344, and can provide feedbacks on the detected pressure information to the suction drive circuit 34 for controlling the suction procedure.

The body 21 is arranged symmetrically with three or four open slots along a circumferential direction, as shown in FIG. 8, where the first open slot 212 and the second open slot 213 are configured to accommodate the power assembly 51 and the suction assembly 52, respectively. The remaining slots, e.g., a third open slot and an optional fourth open slot, are configured to accommodate a fluid-oil compensating system, in order to provide pressure compensation for the power assembly 51 and the suction assembly 52 under high-pressure downhole conditions.

In one embodiment, as shown in FIG. 4, the tank assembly 7 and the fluid-parameter measuring assembly 8 are disposed in the sample drilling tool 25, with both ends secured by a connection block and the dual male connector 26. The tank assembly 7 includes several separate sample tanks 71, preferably consisting of 3 or 4 sample tanks 71. The tank assembly 7 has a drilling-fluid flow pathway 72 at its middle portion, and a shut-off valve 524 and a multiport solenoid valve 81 at its top portion.

The multiport solenoid valve 81 is connected to each of the sample tanks 71, and configured to control the formation fluid 110 to flow to respective sample tanks 71 for storage. The fluid-parameter measuring assembly 8 is connected to an adapter 75 via a fluid-parameter measuring assembly pipeline 82. When the switch valve 524 is open, the formation fluid 110 flows through the pipeline 82 into the fluid-parameter measuring assembly 8, which performs online measurement on viscosity, density and composition of the formation fluid 110, and data storage and upload.

In one embodiment, the online surveying and mapping system includes a pipeline for circulation of drilling fluid. The drilling fluid flows through an instrument compartment carrier pipeline, the sidewall hole 523, an upper line 527, the suction line 522, the liquid channel 519, the flow channel 525, the second carbide impact-resistant ring 344, the first carbide impact-resistant ring 333, the push drive circuit connector line 334, a hydraulic orifice 47, a first pipeline 73, a second pipeline 74, the fluid-parameter measuring assembly pipeline 82, the suction drive circuit connector line 345, and hydraulic pipelines.

In a preferred embodiment, the ground control unit 1 is connected to a ground pulse transmitter 11, a mud pit 12, and a mud pump 13. The ground pulse transmitter 11 emits a pressure pulse signal through a ground pulse generator.

The sampling and survey-and-mapping operations performed by the online survey-and-mapping system of the present embodiment are as follows.

First, the device of the present invention is connected to the drill tool 111, and lowered into the wellbore along with the drill tool 111 during normal drilling operations. When the formation fluid 110 is to be sampled, a sampling command is issued from the ground control unit 1 to control the ground pulse generator to emit a pressure pulse signal.

Upon receiving the sampling command from the ground, the downhole control assembly 9 activates the power portion 5 to work. At this point, the power assembly 51 controls the servo motor 511 to rotate clockwise. The clockwise rotation of the servo motor 511 is transmitted through the coupling 512, the reducer 513, and the bearing pack 514 or the like, to the screw 515, causing it to rotate clockwise. The clockwise rotation of the screw 515 enables, via threads, the power piston 516 to extend out. The power piston 516 transmits power to the push piston 43, which drives the probe assembly 41 to extend into the formation 10, with the other side thereof tightly against the wellbore wall.

After the extension is completed, the suction pump 521 of the suction assembly 52 starts to work. The suction pump draws formation fluid 110 from the formation 10, which is then filtered through the probe filter 42 before entering the contamination-rate evaluating assembly 6.

The contamination-rate evaluating assembly 6 inspects the sample to determine whether it is qualified. Unqualified samples are discharged through the sidewall hole 523, while qualified samples are sent into the sample tank 71 for storage. A portion of qualified sample enters the fluid-parameter measuring assembly 8, for online measurement on the sample of formation fluid 110. After sampling is completed, the servo motor 511 of the power assembly 51 reverses its direction, so that the probe assembly 41 is retracted under the action of the recovery mechanism 44 in the probe assembly, and thus restored to its initial state.

The sample entering the sample tank 71 is measured by the fluid-parameter measuring assembly 8, in order to determine the viscosity, density, and composition of the formation fluid 110, wherein the data is stored and uploaded to the ground.

In the context of the present invention, the upper direction is toward the wellhead, and the lower direction is away from the wellhead.

While the present invention has been described above with reference to the exemplary embodiments, various modifications may be made and components may be replaced with equivalents thereof without departing from the scope of the present invention. In particular, as long as there is no structural conflict, the technical features mentioned in different embodiments can be combined with each other in any manner. The present invention is not limited to the specific embodiments disclosed herein, but includes all technical solutions falling within the scope of the claims.

1. A sampling device, comprising:
a housing comprising a body and a sampling drill collar;
a sampling assembly arranged in the sampling drill collar, comprising a probe assembly, which includes an external probe support mechanism and an internal probe, wherein the probe support mechanism is provided at a bottom thereof with a push piston, for driving the probe to extend in a radial direction relative the sampling drill collar; and
a power assembly and a suction assembly arranged on the body,
wherein the power assembly is configured to push the probe outward in the radial direction into a formation via the push piston, and the suction assembly is configured to draw fluid sample from the formation via the probe, and
the sampling assembly further comprises a recovery mechanism, for driving the probe to retract after drawing.

2. The sampling device according to claim 1, wherein a plurality of recovery mechanisms is uniformly arranged around a circumferential direction of the probe; and
each of the plurality of recovery mechanisms includes an telescopic rod connected to the probe support mechanism and the probe, a recovery spring being arranged externally around the telescopic rod, wherein an end of the recovery spring is connected to the probe and another end thereof is connected to the probe support mechanism.

3. The sampling device according to claim 2, wherein the body is provided with a piston chamber, in which a power piston is provided; and
the piston chamber is filled with hydraulic oil, and has a front end in communication with the push piston, wherein the power piston is moveable in the piston chamber, thereby pumping the hydraulic oil to or away from the push piston.

4. The sampling device according to claim 3, wherein the power assembly further includes a servo motor connected to the power piston via a screw, wherein the servo motor is configured to drive the screw to rotate, so that the screw, when rotating, drives the power piston to extend or retract in the piston chamber via threads.

5. The sampling device according to claim 4, wherein the probe is provided therein with a central hole in communication with a flow channel, and the probe is further provided with a filter.

6. The sampling device according to claim 5, wherein the suction assembly includes a suction pump, which is connected to the central hole of the probe via the flow channel.

7. The sampling device according to claim 6, wherein the power assembly and the suction assembly are powered by a power supply assembly, which includes a rectifier voltage regulator circuit, a main control circuit, a push drive circuit, a suction drive circuit, and an electromagnetic valve control circuit,
wherein the push drive circuit supplies power to the servo motor, and the suction drive circuit supplies power to the suction pump.

8. The sampling device according to claim 7, wherein an end of the probe assembly is provided with a differential pressure sensor connected to the push drive circuit.

9. An online surveying and mapping system, comprising:
a ground control unit for transmitting a control command from ground to downhole; and
a downhole unit, comprising:
a sampling device according to claim 1;
a downhole control assembly, for receiving the control command from the ground control unit to control sampling and online surveying and mapping operations;
a tank assembly, for storing qualified formation samples;
a contamination-rate evaluating assembly, for determining whether a contamination rate of a formation sample is qualified, and discharging unqualified formation sample while delivering qualified formation sample to the tank assembly; and
a fluid-parameter measuring assembly, for measuring parameters of the formation sample in the tank assembly and transmitting measured data to the ground control unit.

10. The online surveying and mapping system according to claim 9, wherein the body is provided at an upper end thereof with an instrument compartment case and an upper case, for installing the downhole control assembly, and at a lower end thereof with the sampling drill collar, wherein the sampling drill collar is provided at a lower end thereof with a sample-measuring drill collar, for installing the fluid-parameter measuring assembly.

11. The online surveying and mapping system according to claim 9, wherein the sampling drill collar is provided with a spiral wing having an outer diameter slightly smaller than wellbore diameter, wherein the spiral wing is provided with a through-hole slot for installing the probe assembly.

12. The online surveying and mapping system according to claim 9, wherein the suction assembly and the contamination-rate evaluating assembly are formed into one piece and connected to the suction pump via a suction line; and
the suction pump is configured to provide negative pressure to the probe via the flow channel for drawing the formation fluid, which flows to the contamination-rate evaluating assembly through the flow channel and the suction line.

13. The online surveying and mapping system according to claim 9, wherein a sidewall of the body is further provided with a sidewall hole communicating the contamination-rate evaluating assembly with an external annulus,
wherein the suction pump pumps the formation sample drawn by the probe assembly to the contamination-rate evaluating assembly, which discharges unqualified formation sample through the sidewall hole and delivers qualified formation sample to the tank assembly.

14. The online surveying and mapping system according to claim 9, wherein the contamination-rate evaluating assembly is connected to the tank assembly via the suction line, the suction pump, the flow channel, a first pipeline and a second pipeline, and an electromagnetic valve or a one-way valve is arranged in each of the flow channel, the first pipeline and the second pipeline.

15. The online surveying and mapping system according to claim 9, wherein the tank assembly and the fluid-parameter measuring assembly are located inside the sample-measuring drill collar, and the fluid-parameter measuring assembly is configured to measure parameters of the formation sample and upload the parameters to the ground control unit.

16. The online surveying and mapping system according to claim 15, wherein the fluid-parameter measuring assembly is connected to an adapter connected to a switch valve via the suction line.
